(19) United States
(12) Patent Application Publication
(10) Pub. No.: US 2026/0275985 A1
(43) Pub. Date: Sep. 17, 2026

(54) COMPRESSOR, METHOD FOR MANUFACTURING COMPRESSOR, AND METHOD FOR EVALUATING COATING MATERIAL FOR COMPRESSOR

(71) Applicant: Carrier Japan Corporation, Tokyo (JP)

(72) Inventor: Yudai HASEGAWA, Shizuoka (JP)

(73) Assignee: Carrier Japan Corporation, Tokyo (JP)

(21) Appl. No.: 19/168,055

(22) PCT Filed: Mar. 27, 2023
(86) PCT No.: PCT/JP2023/012072

§ 371 Date: Sep. 23, 2025

(51) Int. Cl. F04C 23/02; G01N 33/00

(52) U.S. Cl. CPC F04C 23/02; G01N 33/0096; F04C 2230/91; F04C 2240/30; F04C 2240/40

(57) ABSTRACT

A compressor (1) includes a compression mechanism (13) configured to compress a refrigerant, an electric motor configured to drive the compression mechanism, and a casing accommodating the compression mechanism and the electric motor. The refrigerant compressed by the compression mechanism (13) is discharged from the compressor (1) via a space inside the casing, and the compression mechanism (13) is exposed to a high-temperature environment caused by the compressed refrigerant. The casing includes a base material made of a ferrous metal, and the nitrogen gas permeability rate of a coating film formed on an outer surface of the casing is (a) 2,500 [mol·μm/(m²·s·Pa)] or less at −40° C., (b) 150 [mol·μm/(m²·s·Pa)] or less at room temperature, and (c) 2,500 [mol·μm/(m²·s·Pa)] or less at 100° C.

### TECHNICAL FIELD

Embodiments of the present invention relate to a compressor, a method for manufacturing the compressor, and a method for evaluating a coating material used for the compressor.

### BACKGROUND ART

There exists a so-called hermetic-type compressor in which a compression mechanism and an electric motor are accommodated within a sealed casing.

In such hermetic compressors, the refrigerant is compressed by the compression mechanism and then discharged from the compressor through a discharge pipe provided in a cover plate or similar portion of the sealed casing, after passing through the internal space of the casing. During operation of the compressor, the internal space of the sealed casing is filled with compressed refrigerant, which heats the casing from the inside.

### PRIOR ART DOCUMENTS

### Patent Documents

Patent Document 1: Japanese Patent Laid-Open No. 2020-153293

### SUMMARY OF THE INVENTION

### Problems to be Solved by the Invention

In conventional compressors of this type, when selecting a coating material for coating the outer surface of the sealed casing, the primary consideration has been the adhesion between the substrate and the coating film to ensure corrosion resistance by the coating. However, the gas permeability of the coating film has not been adequately considered.

In applications involving refrigeration cycle systems, the sealed casing is not only heated by the compressed refrigerant but also cooled by low-temperature refrigerant returning from the evaporator. In some regions of the casing, the temperature may fall below 0° C.

Therefore, when selecting a coating material for use in compressor applications, it is important to consider not only the adhesion between the substrate and the coating film, but also the gas permeability of the coating film under varying temperature conditions, so that moisture-laden air can be effectively blocked in the actual operating environment of the compressor.

In view of the above, it is an object of the present invention to provide a compressor with enhanced reliability by confirming corrosion resistance of a coating through evaluation based on the gas permeability of the coating film.

### Means for Solving the Problem

A compressor in one aspect of the present invention includes a compression mechanism configured to compress a refrigerant, an electric motor configured to drive the compression mechanism, and a casing accommodating the compression mechanism and the electric motor. In this compressor, the refrigerant compressed by the compression mechanism is discharged from the compressor via a space inside the casing, and the compression mechanism is exposed to a high-temperature environment caused by the compressed refrigerant. The casing includes a base material made of a ferrous metal, and a coating film formed on an outer surface of the casing. The nitrogen gas permeability rate of the coating film is (a) 2,500 [mol·μm/(m²·s·Pa)] or less at −40° C., (b) 150 [mol·μm/(m²·s·Pa)] or less at room temperature, and (c) 2,500 [mol·μm/(m²·s·Pa)] or less at 100° C.

The nitrogen gas permeability rate of a coating film tends to increase not only in high-temperature environments but also in low-temperature environments, relative to a reference temperature near room temperature. In light of this tendency, the coating film formed on the outer surface of the casing is configured to have a nitrogen gas permeability rate of:
(a) 2,500 [mol·μm/m²·s·Pa] or less at −40° C.,
(b) 150 [mol·μm/m²·s·Pa] or less at room temperature, and
(c) 2, 500 [mol·μm/m²·s·Pa] or less at 100° C.

By satisfying these conditions, the gas permeability of the coating film is appropriately adapted to the actual operating environment of the compressor. As a result, the transmission of moisture and oxygen through the coating film can be effectively suppressed across the entire surface of the casing, thereby reducing the occurrence of rust and enabling the provision of a compressor with enhanced reliability.

The compressor may include a low-pressure section configured to receive the refrigerant prior to compression by the compression mechanism and to separate liquid refrigerant contained therein, and a high-pressure section formed separately from the low-pressure section and connected thereto so as to receive the refrigerant after separation of the liquid refrigerant. The casing may include a first casing for the low-pressure section and a second casing for the high-pressure section, and the compression mechanism and the electric motor may be accommodated in the second casing. The coating film may be formed on an outer surface of at least one of the first casing or the second casing.

By dividing the compressor into a low-pressure section and a high-pressure section (formed as separate bodies), and configuring the low-pressure section to separate liquid refrigerant from the refrigerant, while accommodating the compression mechanism and the electric motor within the high-pressure section, it becomes possible to suppress the inflow of liquid refrigerant into the high-pressure section, particularly into the compression mechanism.

In such a compressor, by forming a coating film having the above-described specified gas permeability rate on at least one of the high-pressure section or the low-pressure section, it is possible to suppress the transmission of moisture and other substances not only in areas heated by the compressed refrigerant, but also in areas cooled by the refrigerant returning from the evaporator. As a result, rust formation in both the low-pressure and high-pressure sections can be effectively suppressed.

The coating film may be formed from an epoxy resin having a benzene ring in a portion of the non-crystalline molecular structure backbone and in an additive.

By employing a coating material that combines a highly crosslinked epoxy resin with benzene rings having a rigid molecular structure, it is possible to further reduce gas permeability and thereby promote enhanced anti-corrosion performance through coating.

In another aspect of the present invention, a method for manufacturing a compressor including a casing that accommodates a compression mechanism configured to compress a refrigerant and an electric motor configured to drive the compression mechanism is provided. The method includes a coating-material selection step that selects a coating material for coating an outer surface of the casing. In the coating-material selection step, gas permeability rate of a coating film formed from the coating material is measured at a plurality of temperatures including a first temperature below 0° C., or gas permeability rate exhibited by the coating film at the plurality of temperatures is obtained. A coating material is selected such that the gas permeability rate at each of the plurality of temperatures falls within a predetermined range.

By measuring the gas permeability rate of the coating film during the coating material selection process and conducting selection evaluation based on the results, it becomes possible to verify the anti-corrosion performance of the coating from the perspective of gas permeability and to select a more suitable coating material.

Furthermore, by measuring gas permeability rate at multiple temperatures including a first temperature below 0° C. and evaluating the gas permeability of the coating film under these conditions, the evaluation can be conducted under conditions simulating the actual operating environment of the compressor. This enables effective suppression of rust formation across the entire casing.

In the coating-material selection step, the plurality of temperatures may include the first temperature and room temperature. The coating material may be selected such that the gas permeability rate is equal to or less than a predetermined first threshold at the first temperature, and equal to or less than a predetermined second threshold, which is lower than the first threshold, at room temperature.

By measuring the gas permeability rate at a first temperature and at room temperature, and by setting a second threshold, which is used for comparison with the gas permeability rate at room temperature, lower than a first threshold used for comparison with the gas permeability rate at the first temperature, it becomes possible to reflect temperature-dependent changes in gas permeability rate in the coating selection evaluation. This enables the selection of a more suitable coating material.

The first temperature may be within a range from −40° C. to −20° C.

By setting the first temperature to a value between −40° C. and −20° C., it becomes possible to conduct a more appropriate evaluation that accounts for the tendency of gas permeability rate to increase as temperature decreases. This enables the selection of a coating material that is better suited to low-temperature operating conditions.

In a further aspect of the present invention, a method for evaluating a coating material used for coating a compressor including a casing that accommodates a compression mechanism configured to compress a refrigerant and an electric motor configured to drive the compression mechanism. The method includes preparing a sample formed into a film from the coating material, measuring gas permeability rate of the sample at a plurality of temperatures including a first temperature below 0° C., the gas permeability rate indicating the amount of gas permeating the sample per unit time, and evaluating the performance of the coating film formed by the coating material based on the gas permeability measured at each of the plurality of temperatures.

By preparing a sample in which the coating material is formed into a film, and conducting performance evaluation based on the gas permeability rate of this sample, it becomes possible to verify the anti-corrosion performance of the coating in a relatively simple manner and to select an appropriate coating material.

Furthermore, by measuring gas permeability rate at multiple temperatures including a first temperature below 0° C. and performing performance evaluation under these conditions, the evaluation can be conducted under conditions simulating the actual operating environment of the compressor. This allows for performance evaluation with higher reliability.

### Effects of the Invention

According to the present invention, the anti-corrosion performance of a coating can be appropriately evaluated from the perspective of gas permeability, thereby enabling the provision of a compressor with enhanced reliability.

### BRIEF DESCRIPTION OF THE DRAWINGS

FIG. 1 is a schematic diagram illustrating the configuration of a refrigeration cycle apparatus including a compressor according to an embodiment of the present invention.

FIG. 2 is a cross-sectional view showing the internal structure of the compressor described above.

FIG. 3 is a flowchart illustrating the basic procedure for selecting a coating material.

FIG. 4 is a schematic diagram showing the configuration of a coating film evaluation test apparatus.

FIG. 5 is a schematic diagram showing test results of the differential pressure between the high-temperature chamber and the low-temperature chamber in the evaluation test apparatus, in a high-temperature range above room temperature.

FIG. 6 is a schematic diagram showing test results of the differential pressure between the high-temperature chamber and the low-temperature chamber in the evaluation test apparatus, in a low-temperature range below room temperature.

FIG. 7 is a schematic diagram illustrating the relationship between the nitrogen gas permeability rate of the coating film and temperature.

### DESCRIPTION OF EMBODIMENTS

Referring now to the drawings, embodiments of the present invention will be described in detail.

### (Configuration of the Refrigeration Cycle Apparatus)

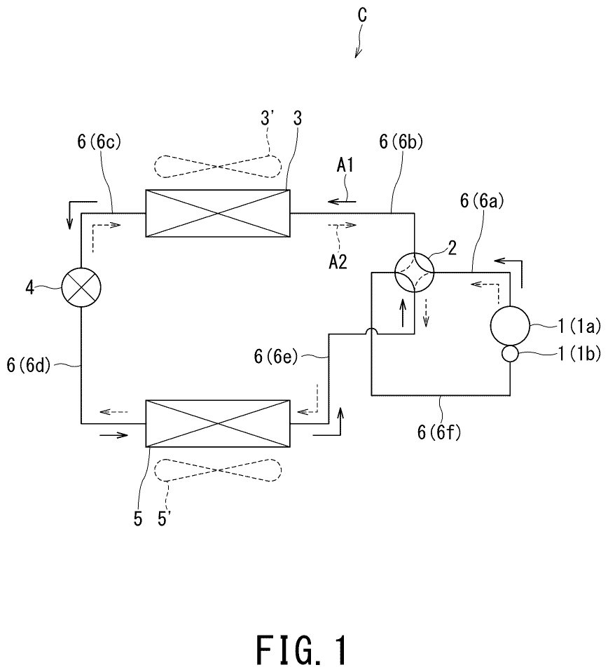

FIG. 1 is a schematic diagram illustrating the configuration of a refrigeration cycle apparatus C including a compressor 1 according to an embodiment of the present invention.

The refrigeration cycle apparatus C is configured as an air conditioner and includes, in addition to the compressor 1, a four-way valve 2, an outdoor heat exchanger 3, an expansion valve 4, and an indoor heat exchanger 5. These refrigeration cycle components 1 through 5 are connected via refrigerant piping 6 (sections 6a to 6f), through which a refrigerant circulates within the apparatus C. Examples of refrigerants applicable to this embodiment include R32, R410A, R448A, R449A, R404A, R407C, R454A, R454B, R454C, and R744.

The operation of the refrigeration cycle apparatus C can be switched between cooling and heating modes by changing the flow path through the four-way valve 2.

### (Cooling Operation)

During cooling operation, the refrigerant circulates along the path indicated by the solid arrows A1 in FIG. 1.

Specifically, the refrigerant discharged from the compressor 1 flows sequentially through the four-way valve 2, the outdoor heat exchanger 3, the expansion valve 4, and the indoor heat exchanger 5 via the refrigerant piping 6. The high-pressure gaseous refrigerant compressed by the compressor 1 is cooled and condensed in the outdoor heat exchanger 3 through heat exchange with ambient air. The outdoor heat exchanger 3 is equipped with an outdoor fan 3′, which introduces outdoor air into the heat exchanger. The condensed refrigerant, now a gas-liquid mixture, passes through the expansion valve 4, where its pressure is reduced, resulting in a low-pressure liquid refrigerant that is supplied to the indoor heat exchanger 5. In the indoor heat exchanger 5, the liquid refrigerant is heated and evaporated through heat exchange with indoor air. The indoor heat exchanger 5 is equipped with an indoor fan 5′, which introduces indoor air into the heat exchanger. The evaporated gas-liquid mixture then returns to the compressor 1 via the four-way valve 2.

### (Heating Operation)

During heating operation, the refrigerant circulates along the path indicated by the dashed arrows A2 in FIG. 1.

Specifically, the refrigerant discharged from the compressor 1 flows sequentially through the four-way valve 2, the indoor heat exchanger 5, the expansion valve 4, and the outdoor heat exchanger 3 via the refrigerant piping 6. The high-pressure gaseous refrigerant compressed by the compressor 1 is cooled and condensed in the indoor heat exchanger 5 through heat exchange with indoor air, thereby releasing heat to the indoor environment. The condensed refrigerant, now a gas-liquid mixture, passes through the expansion valve 4, where its pressure is reduced, resulting in a low-pressure liquid refrigerant that is supplied to the outdoor heat exchanger 3. In the outdoor heat exchanger 3, the liquid refrigerant is heated and evaporated through heat exchange with ambient air, thereby absorbing heat from the outdoor environment. The evaporated gas-liquid mixture then returns to the compressor 1 via the four-way valve 2.

### (Defrost Operation)

During heating operation, when the refrigerant evaporates in the outdoor heat exchanger 3, heat is absorbed from the ambient air, causing water vapor in the air to condense and form droplets that adhere to the heat exchange elements inside the outdoor heat exchanger 3. If the ambient temperature is low, these droplets may freeze and form frost. Frost accumulation can hinder heat exchange and reduce efficiency. Therefore, a defrost operation is performed to remove the frost.

During defrost operation, the four-way valve 2 is set to the same state as in cooling operation, and the refrigerant circulates in the same sequence as during cooling. However, both the outdoor fan 3′ and the indoor fan 5′ are stopped. The high-temperature, high-pressure gaseous refrigerant discharged from the compressor 1 heats the heat exchange elements of the outdoor heat exchanger 3, thereby melting the frost. The resulting water is discharged from the outdoor heat exchanger 3 as drain water.

### (Structure of the Compressor)

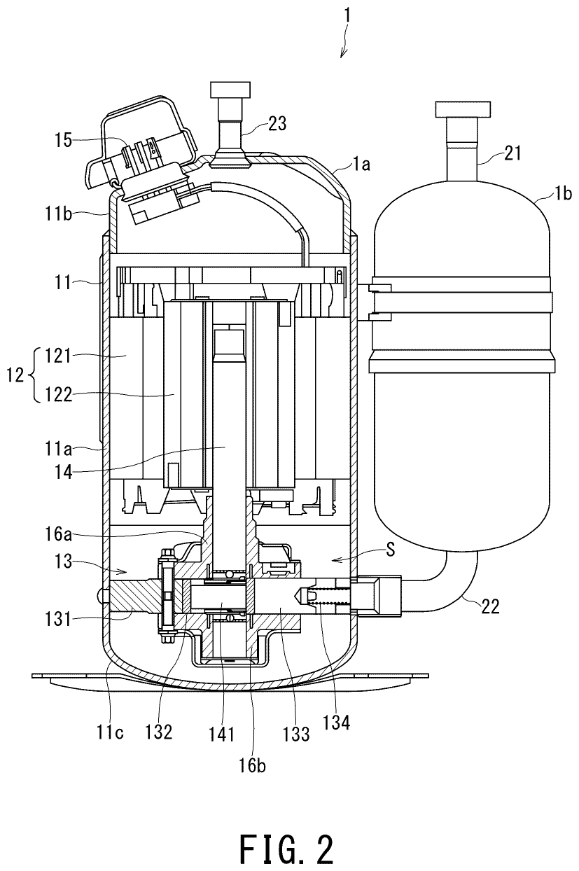

FIG. 2 is a cross-sectional view illustrating the internal structure of the compressor 1 according to the present embodiment.

The compressor 1 includes a compressor body 1a and an accumulator 1b. The accumulator 1b is provided with a suction pipe 21, through which refrigerant discharged from a heat exchanger functioning as an evaporator is supplied to the accumulator 1b. The evaporator corresponds to the indoor heat exchanger 5 during cooling operation or the outdoor heat exchanger 3 during heating operation. The accumulator 1b separates the supplied refrigerant into gas and liquid phases, and supplies the gas-phase refrigerant (i.e., gaseous refrigerant) to the compressor body 1a. In this configuration, the compressor 1 includes the compressor body 1a and the accumulator 1b, which are structurally separated and fluidly connected each other via a refrigerant inlet pipe 22. The compressor body 1a constitutes the “high-pressure section” of the compressor 1, while the accumulator 1b constitutes the “low-pressure section.” The sealed casing 11 of the compressor body 1a corresponds to the “first casing,” and the casing of the accumulator 1b corresponds to the “second casing.”

In this embodiment, the compressor 1 is a hermetic rotary compressor. The compressor 1, particularly the compressor body 1a, includes a sealed casing 11, within which a motor 12 serving as the power source and a compression mechanism 13 are housed. The motor 12 and the compression mechanism 13 are connected via a drive shaft 14, allowing the rotational driving force of the motor 12 to be transmitted to the compression mechanism 13 through the driving shaft 14, thereby enabling the compression mechanism 13 to be driven. Electric power is supplied to the motor 12 from an external power source via a sealed terminal 15. The drive shaft 14 is rotatably supported within the sealed casing 11 by a plurality of bearings, specifically a main bearing 16a and a sub bearing 16b.

The sealed casing 11 is generally cylindrical in shape. It includes a cylindrical casing body 11a open at top and bottom ends, which are sealed by hemispherical end plates 11b and 11c. The casing body 11a and the lower end plate 11c may be integrally formed. The refrigerant inlet pipe 22 extending from the accumulator 1b is connected to the casing body 11a, allowing the gas-phase refrigerant separated by the accumulator 1b to be introduced into the interior of the sealed casing 11. After compression by the compression mechanism 13, the gaseous refrigerant passes through the internal space S of the sealed casing 11, specifically, the space excluding the volume occupied by the motor 12, compression mechanism 13, and drive shaft 14 within the entire interior of the sealed casing 11, and is discharged to the outside via a discharge pipe 23 mounted on the upper end plate 11b.

Inside the sealed casing 11, the motor 12 is housed in the upper half of the casing body 11a, while the compression mechanism 13 is housed in the lower half.

The motor 12 includes a stator 121 fixed to the casing body 11a and a rotor 122 disposed within the inner diameter of the stator 121 and fixed to the drive shaft 14. The rotor 122 is rotatable relative to the stator 121 together with the drive shaft 14. The motor 12 is a permanent magnet synchronous motor, with coils formed on the stator 121 and permanent magnets mounted or embedded in the rotor 122. By energizing the coils, the rotor 122 is rotated, thereby driving the compression mechanism 13 via the drive shaft 14.

The compression mechanism 13 is disposed between the main bearing 16a and the sub bearing 16b. In this embodiment, the compression mechanism 13 is a single-stage compression mechanism including a cylinder 131, an eccentric portion 141 of the drive shaft 14, and a rolling piston 132. The number of compression stages is not limited to one and may be multiple.

The cylinder 131 defines a cylinder chamber, and the rolling piston 132, which is annular in shape, is loosely fitted onto the eccentric portion 141 and housed together with the eccentric portion 141 in the cylinder chamber. The inner diameter of the cylinder 131 is larger than the outer diameter of the rolling piston 132, forming a clearance therebetween.

The cylinder 131 is provided with a vane groove extending radially from the inner peripheral portion of the cylinder 131, and a sliding vane 133 is inserted into this groove. A spring 134 is compressed between the cylinder 131 and the sliding vane 133, pressing the sliding vane 133 against the outer peripheral surface of the rolling piston 132.

The clearance between the cylinder 131 and the rolling piston 132 is divided by the sliding vane 133 into two chambers: a suction chamber and a compression chamber. The suction chamber and compression chamber of the compression mechanism 13 are sealed by the main bearing 16a located on the upper side and the sub bearing 16b located on the lower side.

An intake port is formed in the cylinder 131, opening to the inner peripheral surface of the cylinder 131 and connected to the suction chamber. The refrigerant inlet pipe 22 extending from the accumulator 1b is inserted into this intake port, allowing the refrigerant discharged from the accumulator 1b to be introduced into the suction chamber via the intake port.

Lubricating oil is sealed within the sealed casing 11, and the compression mechanism 13 is mostly immersed in this oil. An example of a suitable lubricating oil for this embodiment is a polyol ester lubricant containing 0.5 wt % glycidyl ester epoxy and 0.1 wt % phenolic antioxidant.

The casings of the compressor 1, specifically, the sealed casing 11 of the compressor body 1a and the casing of the accumulator 1b, respectively include a base material made of a ferrous metal. Suitable materials for the base material of these casings include steel sheets, specifically hot-rolled steel sheets (SPHE grade), and in this embodiment, SPHE-P grade is employed. To protect against rust and other corrosion, the base material of the casing is subjected to appropriate plating treatments such as electro-galvanizing, followed by coating to form a coating film on the outer surface. As for the actual situation regarding the coating of compressors, when selecting a coating material for coating the outer surface of the sealed casing, adhesion between the base material and the coating film would be primarily considered, while gas permeability of the coating film is generally not taken into account.

During operation of the compressor 1, the internal space of the sealed casing 11 is filled with compressed refrigerant, and the sealed casing 11 is heated by the compressed refrigerant. Meanwhile, the casing of the accumulator 1b is expected to be cooled to temperatures below 0° C. by the relatively low-temperature refrigerant returning from the evaporator. Similarly, certain areas of the casing of the compressor body 1a or the sealed casing 11, such as the joint portion where the refrigerant inlet pipe 22 is connected and its surroundings, may also be cooled to temperatures below 0° C.

Accordingly, in selecting the coating material used for coating the compressor 1, consideration is given not only to the adhesion between the base material and the coating film, but also to the gas permeability of the coating film in response to temperature variations. This approach effectively suppresses the permeation of moisture-laden air under actual operating conditions of the compressor 1, thereby ensuring the required gas barrier properties not only under normal ambient conditions at room temperature, but also in heated environments caused by compressed refrigerant and in cooled environments resulting from refrigerant returning from the evaporator. In this embodiment, the selection of the coating material is carried out in accordance with the procedure described below.

### (Selection Process for Coating Materials)

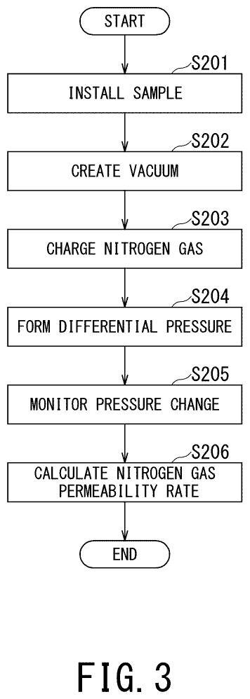

FIG. 3 is a flowchart illustrating the basic procedure for selecting the coating material used in the present embodiment.

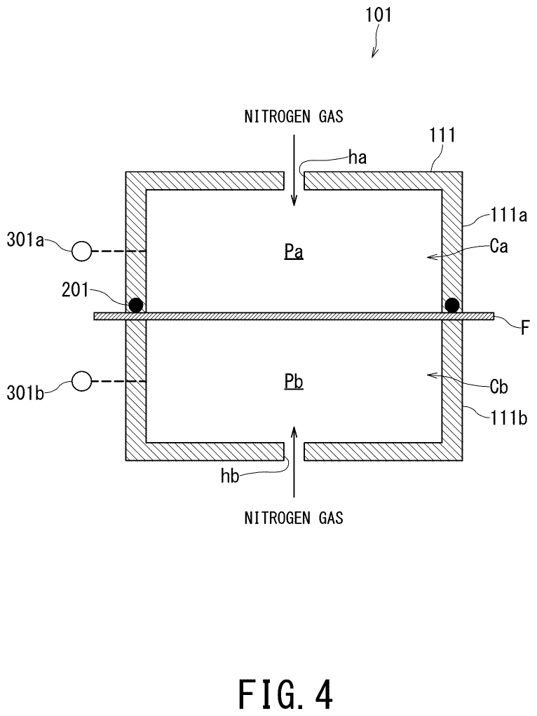

FIG. 4 is a schematic diagram showing the configuration of a coating film evaluation test apparatus 101.

Referring to FIG. 4 as appropriate, the coating film selection process applicable to the manufacturing of compressor 1 in the present embodiment will be described primarily with reference to FIG. 3.

The coating film selection process of the present embodiment includes a step of measuring the nitrogen gas permeability rate R of the coating film. Although the primary targets for barrier performance of the coating film in terms of rust prevention are oxygen and moisture present in the atmosphere, nitrogen gas is employed as the test gas due to its demonstrated correlation with oxygen and moisture in terms of gas permeability characteristics. The measurement of nitrogen gas permeability rate R is conducted in accordance with Japanese Industrial Standard (JIS) K7126-1 or International Standard ISO 15105-2, specifically using the differential pressure method defined in JIS K7126-1.

In step S201, a sample F of the coating film is prepared and installed in the evaluation test apparatus 101.

The sample F is formed by creating a film of the coating material with a predetermined thickness (e.g., 130 μm). Specifically, after applying a release agent to a mold having an appropriate shape, a coating material to be tested, or a test material, is baked onto the mold to achieve curing. Subsequently, the resulting coating film is separated from the mold, and additional layers of the test material are then applied to the released film as many times as necessary, to obtain a sample F with the predetermined thickness. Baking is employed in preparing the coating film to simulate the production coating process, which involves baking.

As shown in FIG. 4, the evaluation test apparatus 101 includes a container 111 that is separable into an upper half 111a and a lower half 111b. The interior of the container 111 is divided into a high-pressure-side space, or a high-pressure chamber Ca, and a low-pressure-side space, or a low-pressure chamber Cb, by the sample F clamped between the upper and lower halves. That is, in this embodiment, the interior of the upper half 111a forms the high-pressure chamber Ca, and the interior of the lower half 111b forms the low-pressure chamber Cb. An appropriate sealing member 201, such as an O-ring, is interposed between the sample F and the upper half 111a.

The upper half 111a is provided with a first vacuum/gas inlet port ha, extending through the upper half 111a from the interior to the exterior. Similarly, the lower half 111b is provided with a second vacuum/gas inlet port hb, extending through the lower half 111b from the interior to the exterior. Through the first vacuum/gas inlet port ha, residual gases remaining inside the high-pressure chamber Ca can be evacuated, and a predetermined test gas can be introduced. Through the second vacuum/gas inlet port hb, residual gases remaining in the low-pressure chamber Cb can be evacuated, and a predetermined test gas can be introduced. Specifically, a vacuum pump and a first gas supply unit are connected to the high-pressure chamber Ca via the first vacuum/gas inlet port ha, and a vacuum pump and a second gas supply unit are connected to the low-pressure chamber Cb via the second vacuum/gas inlet port hb. In this embodiment, nitrogen gas is used as the test gas. Pressure sensors 301a and 301b are respectively connected to the upper half 111a and the lower half 111b, in a manner that allows detection of the pressures Pa and Pb in the high-pressure chamber Ca and the low-pressure chamber Cb, respectively.

In step S202, residual gases are evacuated from both the high-pressure chamber Ca and the low-pressure chamber Cb to create a vacuum in each chamber Ca, Cb. This removes dissolved gases from the sample F and expels them from each chamber Ca, Cb along with residual gases.

In step S203, nitrogen gas is introduced into the high-pressure chamber Ca and the low-pressure chamber Cb until atmospheric pressure is reached.

In step S204, a differential pressure Pd is created between the high-pressure chamber Ca and the low-pressure chamber Cb. Specifically, after the pressures in chambers Ca and Cb have reached atmospheric pressure, nitrogen gas is further introduced into the high-pressure chamber Ca. In this embodiment, the differential pressure Pd is set to 0.020 MPa. The differential pressure Pd may be adjusted as appropriate to avoid deformation of the sample F installed in the apparatus 101.

Alternatively, the differential pressure Pd may be formed by introducing nitrogen gas into the high-pressure chamber Ca until the desired pressure (i.e., 0.020 MPa in this embodiment) is reached, after vacuum evacuation in each chamber Ca, Cb.

In step S205, the change in pressure in the high-pressure chamber Ca is monitored over a predetermined period of time. In the present embodiment, the duration for monitoring pressure change is approximately ten hours. As nitrogen gas in the high-pressure chamber Ca permeates through the sample F into the low-pressure chamber Cb, the pressure in the high-pressure chamber Ca decreases. Since the pressure in the low-pressure chamber Cb is maintained at atmospheric pressure, the pressure change in the high-pressure chamber Ca reflects the change in differential pressure Pd between the high-pressure chamber Ca and the low-pressure chamber Cb.

In step S206, the nitrogen gas permeability rate R of the sample F is calculated based on the change in pressure in the high-pressure chamber Ca, or the change in differential pressure between the high-pressure and low-pressure chambers Ca and Cb. The calculation of the nitrogen gas permeability rate R is performed using the following equations:

$$μ = \{ Vc / ( R \cdot T \cdot Pu \cdot A ) \} \cdot dP / dt \qquad ( 1 )$$

$$Q = μ \cdot h \qquad ( 2 )$$

Equation (1) represents the gas permeability [mol/(m²·s·Pa)], and equation (2) represents the gas permeation coefficient [mol·μm/(m²·s·Pa)]. Here, Vc is the volume of the high-pressure chamber Ca [m³], R is the gas constant, T is the test temperature [K], Pu is the differential pressure [Pa], A is the gas permeation area [m²], dP/dt is the pressure change rate [Pa/s], and h is the thickness of the sample F [μm]. In this embodiment, the gas permeation coefficient Q of nitrogen gas is used as the nitrogen gas permeability rate R, and the performance of the coating film is evaluated from the perspective of nitrogen gas permeability. As previously described, in a situation where the low-pressure chamber Cb is maintained in a vacuum state and nitrogen gas is introduced into the high-pressure chamber Ca at a predetermined pressure, it is also possible to monitor pressure changes in the low-pressure chamber Cb and calculate the nitrogen gas permeability rate R based on the pressure in the low-pressure chamber Cb.

In this embodiment, the procedure shown in FIG. 3 is carried out at multiple test temperatures T, including temperatures below 0° C. In this embodiment, the test temperatures T include −40° C., −20° C., −10° C., 0° C., 25° C., 40° C., 50° C., 70° C., and 106° C. An epoxy resin having benzene rings in a portion of non-crystalline molecular structure backbone and in additives is employed as the coating material. Here, 25° C. is considered room temperature, and 106° C. corresponds to the glass transition temperature of the coating film formed from the epoxy resin.

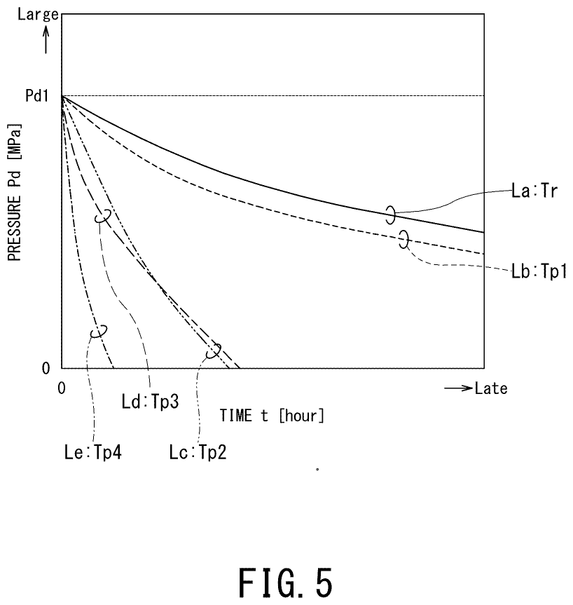

FIG. 5 is a schematic diagram showing the test results of the variation in differential pressure Pd between the high-temperature chamber Ca and the low-temperature chamber Cb in the evaluation test apparatus 101, for each of the test temperatures T at or above room temperature.

In FIG. 5, the horizontal axis represents time t, and the vertical axis represents the differential pressure Pd. The bold solid line La indicates the differential pressure Pd at the test temperature Tr=25° C. The dotted line Lb indicates the differential pressure Pd at the test temperature Tp1=40° C., the two-dot chain line Lc indicates the differential pressure Pd at the test temperature Tp2=50° C., the long dashed line Ld indicates the differential pressure Pd at the test temperature Tp3=70° C., and the one-dot chain line Le indicates the differential pressure Pd at the test temperature Tp4=106° C., respectively.

Under high-temperature conditions above room temperature Tr, the differential pressure Pd at Tp1=40° C. is approximately equivalent to that at the reference room temperature Tr, or slightly lower. This indicates that there is no significant change in the nitrogen gas permeability rate R between Tr and Tp1=40° C. In contrast, at Tp2=50° C., the differential pressure Pd decreases significantly compared to Tp1=40° C., suggesting that the nitrogen gas permeability rate R begins to increase as the temperature rises from Tp1=40° C. to Tp2=50° C. At Tp2=50° C. and Tp3=70° C., the differential pressure Pd remains substantially unchanged. However, at Tp4=106° C., which corresponds to the glass transition temperature, the differential pressure Pd decreases sharply again, indicating a corresponding sharp increase in the nitrogen gas permeability rate R.

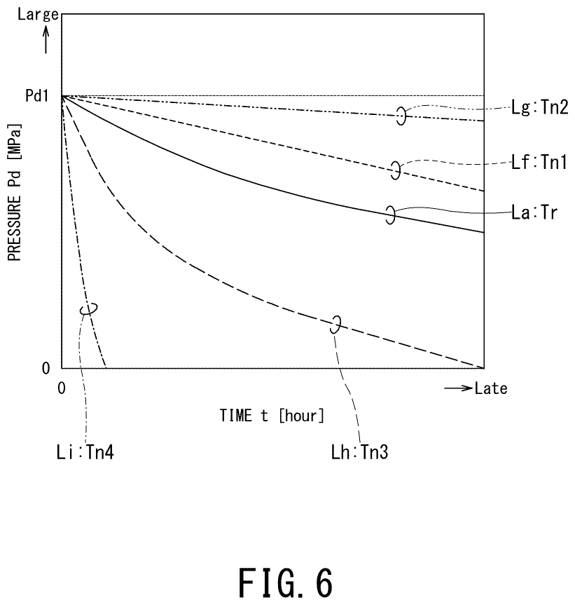

FIG. 6 is a schematic diagram showing the test results of the variation in differential pressure Pd between the high-temperature chamber Ca and the low-temperature chamber Cb in the evaluation test apparatus 101, for each test temperature T below room temperature.

In FIG. 6, the horizontal axis represents time t, and the vertical axis represents the differential pressure Pd. The bold solid line La indicates the differential pressure Pd at the test temperature Tr=25° C. The dotted line Lf indicates the differential pressure Pd at the test temperature Tn1=0° C. A two-dot chain line Lg indicates the differential pressure Pd at the test temperature Tn2=−10° C. A long dashed line Lh indicates the differential pressure Pd at the test temperature Tn3=−20° C. A one-dot chain line Li indicates the differential pressure Pd at the test temperature Tn4=−40° C., respectively.

Under low-temperature conditions below room temperature Tr, the decrease in differential pressure Pd becomes more gradual as the test temperature T decreases from room temperature Tr=25° C. to Tn2=−10° C. This indicates that the nitrogen gas permeability rate R tends to decrease at least down to Tn2=−10° C. In contrast, at Tn3=−20° C., the differential pressure Pd is lower than that at room temperature Tr=25° C., suggesting that the nitrogen gas permeability rate R begins to increase as the temperature decreases from Tn2=−10° C. to Tn3=−20° C. Furthermore, at Tn4=−40° C., the differential pressure Pd decreases sharply, indicating a corresponding sharp increase in the nitrogen gas permeability rate R.

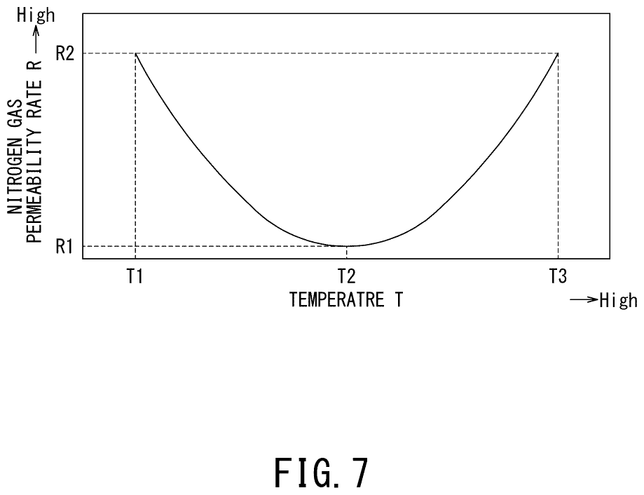

FIG. 7 is a schematic diagram summarizing the relationship between the nitrogen gas permeability rate R of the coating film and the temperature T. The horizontal axis represents the temperature T, and the vertical axis represents the nitrogen gas permeability rate R.

Based on the experimental results shown in FIGS. 4 and 5, the nitrogen gas permeability rate R exhibits its minimum value at room temperature Tr=25° C. or at a temperature near this value, such as approximately Tn1=0° C. As the temperature T increases from around Tp1=40° C. toward Tp4=106° C., the nitrogen gas permeability rate R increases accordingly. On the other hand, the nitrogen gas permeability rate R begins to increase when the temperature drops below Tn2=−10° C., and continues to increase as the temperature T decreases toward Tn4=−40° C. FIG. 7 schematically illustrates the relationship between temperature T and nitrogen gas permeability rate R, organized as a generalized correlation based on the above-described results.

In this embodiment, multiple evaluation temperatures T1 to T3 are adopted, including a temperature T1 below 0° C., for assessing the nitrogen gas permeability rate R of the coating film. Specifically, considering the actual operating environment of the compressor 1 in the refrigeration cycle apparatus C, the temperatures T1=−40° C., T2=25° C., and T3=100° C. are employed. Temperature T1=−40° C. simulates the cooling environment caused by the refrigerant returning from the evaporator; T2=25° C. represents a simulated normal condition under room temperature; and T3=100° C. simulates the heating environment caused by the compressed refrigerant. These temperatures T1 to T3 can be appropriately set according to the actual operating conditions of the compressor 1, that is, the temperatures that the refrigerant may reach during operation. Here, temperature T1 corresponds to the “first temperature” in this embodiment.

In step S206 of the flowchart shown in FIG. 3, the nitrogen gas permeability rate R is measured at each of the test temperatures T1=−40° C., T2=25° C., and T3=100° C., and the performance of the coating film is evaluated. Specifically, a sample is selected if it satisfies the following conditions:
(a) the nitrogen gas permeability rate R at T1=−40° C. is equal to or less than 2500 [mol·μm/(m²·s·Pa)];
(b) the nitrogen gas permeability rate R at T2=25° C. is equal to or less than 150 [mol·μm/(m²·s·Pa)]; and
(c) the nitrogen gas permeability rate R at T3=100° C. is equal to or less than 2500 [mol·μm/(m²·s·Pa)].

The coating material of the selected sample is then designated as the coating material to be used for coating the compressor 1. Here, in this embodiment, R=2500 [mol·μm/(m²·s·Pa)] corresponds to the “first threshold” of gas permeability rate, and R=150 [mol·μm/(m²·s·Pa)] corresponds to the “second threshold” of gas permeability rate.

After selecting the coating material, the compressor 1, specifically the outer surfaces of the sealed casing of the compressor body 1a and the casing of the accumulator 1b, is coated using a powder coating method, and assembly of the compressor 1 is then carried out. The assembly process following the coating can be carried out in accordance with conventional procedures.

### Description of Advantages and Effects

In this embodiment, the nitrogen gas permeability rate R of the coating film formed on the casing of the compressor 1, specifically the outer surfaces of the compressor body 1a and the accumulator 1b, is: (a) 2,500 [mol·μm/(m²·s·Pa)] or less at −40° C., (b) 150 [mol·μm/(m²·s·Pa)] or less at room temperature, and (c) 2,500 [mol·μm/(m²·s·Pa)] or less at 100° C. This configuration effectively suppresses the permeation of moisture and oxygen through the coating film under actual operating conditions of the compressor 1, thereby preventing rust formation on the base material made of ferrous metal across the entire casing, and enabling the provision of a highly reliable compressor 1.

By dividing the compressor 1 into a low-pressure section (accumulator 1b) and a high-pressure section (compressor body 1a), and forming the coating film having the above-specified nitrogen gas permeability rate R on at least one of the casing, i.e., the casing of the low-pressure section (casing of the accumulator 1b) or the casing of the high-pressure section (sealed casing 11 of the compressor body 1a), it becomes possible to effectively suppress not only the permeation of moisture at heated portions due to compressed refrigerant, but also at cooled portions due to refrigerant returning from the evaporator. As a result, rust formation in both the low-pressure and high-pressure sections can be effectively prevented.

By employing a specific coating material composed of a highly crosslinked epoxy resin, or an epoxy resin exhibiting a high crosslink density, combined with benzene rings having a rigid molecular structure, the gas permeability can be further reduced, thereby enhancing the anti-corrosion performance of the coating.

By measuring the gas permeability rate of the coating film in the selection of coating materials used for coating the casing of the compressor 1 and evaluating performance based on these measurements, the anti-corrosion performance of the coating can be verified from the perspective of gas permeability. This enables the selection of more suitable coating materials. Using nitrogen gas as the test gas ensures correlation in the gas permeability with the actual target gases, oxygen and moisture, allowing for safe and appropriate performance evaluation.

Furthermore, by measuring the gas permeability rate of the coating film under multiple temperature conditions, including a temperature (first temperature) below ° C., and evaluating the gas permeability of the coating material, it is possible to evaluate the gas permeability under conditions simulating the actual operating environment of the compressor 1. This contributes to suppressing rust formation on the casing across the entire compressor 1. Additionally, by evaluating the adhesion between the base material and the coating film along with gas permeability, further improvements in coating performance can be achieved.

By measuring the gas permeability rate at both the first temperature and room temperature, and setting the second threshold, used for comparison with the gas permeability rate at room temperature, lower than the first threshold, used for comparison with the gas permeability rate at the first temperature, it becomes possible to reflect temperature-dependent changes in gas permeability rate in the performance evaluation of the coating film, thereby enabling the selection of more appropriate coating materials.

By setting the first temperature within a range of −40° C. to −20° C., a more appropriate evaluation can be conducted that takes into account the tendency of gas permeability rate, specifically the tendency of nitrogen gas permeability rate R, to increase as temperature decreases.

Furthermore, by preparing a sample F of the coating film formed into a film shape and conducting performance evaluation based on its gas permeability rate, the anti-corrosion performance of the coating can be verified through a relatively simple method, thereby facilitating the selection of suitable coating materials.

A number of embodiments of the present invention have thus been described; however, those embodiments are presented as examples and are not intended to limit the scope of the invention. It is possible to carry out the novel embodiments in other various modes and to apply various omissions, substitutions, and changes thereto without departing from the gist of the invention. The embodiments and modifications thereof are included in the scope and the gist of the invention, and are also included in the scope of the invention set forth in the claims and equivalents thereof.

### REFERENCE SIGNS LIST

C: refrigeration cycle apparatus, 1: compressor, 1a: compressor body, 1b: accumulator, 11: sealed casing, 11a: casing body, 11b, 11c: end plate, 12: motor, 121: stator, 122: rotor, 13: compression mechanism, 131: cylinder, 132: rolling piston, 133: sliding vane, 134: spring, 14: drive shaft, 15: sealed terminal, 16a: main bearing, 16b: sub bearing, 21: suction pipe, 22: refrigerant inlet pipe, 23: discharge pipe, 2: four-way valve, 3: outdoor heat exchanger, 3′: outdoor fan, 4: expansion valve, 5: indoor heat exchanger, 5′: indoor fan, 6, 6a to 6f: refrigerant piping, S: internal space of sealed casing.

1. A compressor comprising:
a compression mechanism configured to compress a refrigerant;
an electric motor configured to drive the compression mechanism; and
a casing accommodating the compression mechanism and the electric motor,
wherein the refrigerant compressed by the compression mechanism is discharged from the compressor via a space inside the casing, and the compression mechanism is exposed to a high-temperature environment caused by the compressed refrigerant,
wherein the casing includes:
a base material made of a ferrous metal; and
a coating film formed on an outer surface of the casing,
wherein the nitrogen gas permeability rate of the coating film is:
(a) 2,500 [mol·μm/(m²·s·Pa)] or less at −40° C.,
(b) 150 [mol μm/(m²·s·Pa)] or less at room temperature, and
(c) 2,500 [mol μm/(m²·s·Pa)] or less at 100° C.

2. The compressor according to claim 1, further comprising:
a low-pressure section configured to receive the refrigerant prior to compression by the compression mechanism and to separate liquid refrigerant contained therein; and
a high-pressure section formed separately from the low-pressure section and connected thereto so as to receive the refrigerant after separation of the liquid refrigerant,
wherein the casing includes a first casing for the low-pressure section and a second casing for the high-pressure section,
wherein the compression mechanism and the electric motor are accommodated in the second casing, and
wherein the coating film is formed on an outer surface of at least one of the first casing or the second casing.

3. The compressor according to claim 1,
wherein the coating film is formed from an epoxy resin including benzene rings in a portion of non-crystalline molecular structure backbone and in an additive.

4. A method for manufacturing a compressor including a casing that accommodates a compression mechanism configured to compress a refrigerant and an electric motor configured to drive the compression mechanism, the method comprising:
selecting a coating material for coating an outer surface of the casing, the selecting comprising:
measuring a gas permeability rate of a coating film formed from the coating material at a plurality of temperatures including a first temperature below 0° C., or
obtaining the gas permeability rate exhibited by the coating film at the plurality of temperatures, and
selecting the coating material such that the gas permeability rate at each of the plurality of temperatures falls within a predetermined range.

5. The method for manufacturing a compressor according to claim 4, wherein,
the plurality of temperatures includes the first temperature and room temperature, and
the coating material is selected such that the gas permeability rate is equal to or less than a predetermined first threshold at the first temperature, and equal to or less than a predetermined second threshold, which is lower than the first threshold, at room temperature.

6. The method for manufacturing a compressor according to claim 5,
wherein the first temperature is within a range from −40° C. to −20° C.

7. A method for evaluating a coating material used for coating a compressor including a casing that accommodates a compression mechanism configured to compress a refrigerant and an electric motor configured to drive the compression mechanism, the method comprising:
preparing a sample formed into a film from the coating material;
measuring a gas permeability rate of the sample at a plurality of temperatures including a first temperature below 0° C., the gas permeability rate indicating the amount of gas permeating the sample per unit time; and
evaluating a performance of the coating film formed from the coating material based on the gas permeability rate measured at each of the plurality of temperatures.

8. The compressor according to claim 2,
wherein the coating film is formed from an epoxy resin including benzene rings in a portion of non-crystalline molecular structure backbone and in an additive.
